(19) United States
(12) Patent Application Publication
(10) Pub. No.: US 2006/0222587 A1
(43) Pub. Date: Oct. 5, 2006

(54) Hybrid inorganic nanoparticles, methods of using and methods of making

(76) Inventors: Paras Prasad, Williamsville, NY (US); Richard V. Mazurchuk, Clarence Center, NY (US); Dhruba J. Bharali, Amherst, NY (US); Dinesh K. Sukumaran, East Amherst, NY (US); Earl J. Bergey, South Dayton, NY (US); Joseph A. Spernyak, West Seneca, NY (US); Mukund Seshadri, Buffalo, NY (US)

Correspondence Address: ROGALSKY & WEYAND, LLP, P.O. BOX 44, LIVONIA, NY, 14487, US

(21) Appl. No.: 11/392,057

(22) Filed: Mar. 29, 2006

Related U.S. Application Data
(60) Provisional application No. 60/666,114, filed on Mar. 29, 2005.

(51) Int. Cl. A61K 51/00

(52) U.S. Cl. 424/1.11; 977/927

(57) ABSTRACT

The invention provides hybrid inorganic nanoparticles, methods of making the hybrid inorganic nanoparticles and methods of using the hybrid inorganic nanoparticles.

The present application claims priority to U.S. Provisional Application No. 60/666,114, filed Mar. 29, 2005, which is hereby incorporated by reference herein.

### FIELD OF THE INVENTION

The subject invention is directed generally to hybrid inorganic nanoparticles, methods of making hybrid inorganic nanoparticles and methods of using the hybrid inorganic nanoparticles.

### BRIEF DESCRIPTION OF THE DRAWINGS

These and other features and advantages of this invention will be evident from the following detailed description of preferred embodiments when read in conjunction with the accompanying drawings in which:

FIG. 1 is a representative ¹⁹F spectra of silica based TFMPTS nanoparticles at 376.3 MHz.

FIG. 2 illustrates a typical ¹⁹F spectra obtained from neat silica based TFPTMS ¹⁹F containing nanoparticles immediately before imaging at 188.34 MHz.

FIG. 3 illustrates ¹⁹F spectra obtained from neat silica based TFPTMS ¹⁹F containing nanoparticles as compared to 1000 mM sodium fluoride (NaF) in aqueous solution at 188.34 MHz.

FIG. 4 depicts representative ¹H and ¹⁹F MR images following administration of TFMPTS nanoparticles in a mouse. ¹H MR images obtained at 200 MHz. (A, axial; B, coronal) and ¹⁹F MR images obtained at 188 MHz. (C, axial; D, coronal) obtained immediately after oral administration of silica based TFPTMS nanoparticles. As shown, arrows denote location of stomach (A, B, E and F) spinal canal (A); lung (B); lobe of liver (B).

FIG. 5 is a ¹⁹F MR image of semi-solid crystalline aggregates of TFPTMS ¹⁹F nanoparticles (left panel, scale denotes 1 cm) and corresponding micrograph (right panel) of nanoparticles in a glass vial photographed using a surgical microscope with attached Nikon 1.2 Mb digital camera (Nikon CoolPix 950, Nikon, USA).

### DETAILED DESCRIPTION OF THE INVENTION

Throughout this application various publications are referenced, many in parenthesis. Full citations for each of these publications are provided at the end of the Detailed Description. The disclosures of each of these publications in their entireties are hereby incorporated by reference in this application.

The subject invention provides hybrid inorganic nanoparticles, methods of making the hybrid inorganic nanoparticles and methods of using the hybrid inorganic nanoparticles.

As used herein, “hybrid inorganic” nanoparticles refer to nanoparticles which contain both organic and inorganic groups. Although not meaning to be bound by theory, the nanoparticles of the invention have the desirable physical properties of both ceramic materials and the functional groups associated with the nanoparticles.

Further, as used herein, the hybrid inorganic nanoparticles of the present invention are used in spectroscopic and image based acquisitions, including, but not limited to, magnetic resonance, fluorescence, bioluminescence spectroscopy and other imaging techniques and other biomedical applications.

The nanoparticles of the present invention are hybrid inorganic nanoparticles which include ¹⁹F nuclei. In one embodiment of the invention, the nanoparticles are silica based hybrid inorganic nanoparticles. The nanoparticles are constructed having various diameters and distribution ranging from about 20 nanometers to about 200 nanometers, and all ranges therein. For example, in one embodiment of the invention, the hybrid inorganic nanoparticles are from about 50 to about 200 nanometers in diameter. In alternative embodiments of the present invention, the nanoparticles are from about 100 to about 200 nanometers, from about 150 to about 200 nanometers or from about 75 to about 200 nanometers. In another embodiment, the nanoparticles are from about 20 nanometers to less than about 200 nanometers, for example from about 20 nanometers, up to about 50, 75, 100 or 150 nanometers.

The nanoparticles of the present invention have a high number of ¹⁹F nuclei per nanoparticle. As used herein, a high number is defined as having up to about 600,000 ¹⁹F nuclei per nanoparticle. In one embodiment, the nanoparticles of the present invention include from about 2000 to about 600,000 ¹⁹F nuclei per nanoparticle. In one embodiment, the nanoparticles have from 10,000 to about 600,000 ¹⁹F nuclei per nanoparticle. In one embodiment, the nanoparticles include from about 30,000 to about 600,000 ¹⁹F nuclei per nanoparticle, or from about 100,000 to about 600,000 ¹⁹F nuclei per nanoparticle.

Therefore, the nanoparticles of the present invention include a quantity of ¹⁹F nuclei to be used in the methods of the present invention, for example, in imaging, spectroscopic acquisitions and biomedical applications.

Although not meaning to be bound by theory, the number of ¹⁹F nuclei per nanoparticle may be calculated by first determining the size of each nanoparticle. For each size of nanoparticle, the mass of the nanoparticle can be determined, and, accordingly, because the mass of each molecule present in each nanoparticle is known, the resultant number of molecules present in the nanoparticle can be calculated by one skilled in the art. For example, a nanoparticle of the present invention having a diameter of about 40 nanometers has approximately 105,000 molecules present in the nanoparticle. Each molecule of the nanoparticle has about three fluorine atoms contained therein. Assuming approximately 30%-40% incorporation of ¹⁹F nuclei per nanoparticle, a nanoparticle having a diameter of approximately 40 nanometers would have about 105,000 ¹⁹F nuclei per nanoparticle. Using these calculations, a nanoparticle having about a 20 nanometer diameter would have approximately 13,000 ¹⁹F nuclei per nanoparticle. Likewise, a nanoparticle having about a 100 nanometer diameter would have approximately 273,000 ¹⁹F nuclei per nanoparticle and a nanoparticle having about a 200 nanometer diameter would have approximately 600,000 ¹⁹F nuclei per nanoparticle.

In one aspect of the invention, the ¹⁹F nuclei are contained in the inner core of the nanoparticles. In an alternative embodiment, the ¹⁹F nuclei are contained at the outer surface of the nanoparticles. In another alternative embodiment, the ¹⁹F is contained both in the inner core and at the outer surface of the nanoparticles.

In another aspect of the invention, the nanoparticles additionally include a biomarker, such as a fluorescent dye, bioluminescent marker and/or near infrared (NIR) marker.

In another aspect of the invention, the nanoparticles include a therapeutic or diagnostic agent, or both. The therapeutic and diagnostic agents are either hydrophilic or hydrophobic. Therapeutic or diagnostic agents include substances capable of treating or preventing an infection systemically or locally, as, for example, antibacterial agents such as penicillin, cephalosporins, bacitracin, tetracycline, doxycycline, quinolines, clindamycin, and metronidazole; antiparasitic agents such as quinacrine, chloroquine and vidarabine; antifungal agents such as nystatin; antiviral agents such as acyclovir, ribarivin and interferons; anti-inflammatory agents such as hydrocortisone and prednisone; analgesic agents such as salicylic acid, acetaminophen, ibuprofen, naproxen, piroxicam, flurbiprofen and morphine; local anesthetics such as lidocaine, bupivacaine, benzocaine, and the like; immunogens (vaccines) for stimulating antibodies against hepatitis, influenza, measles, rubella, tetanus, polio and rabies; peptides such as leuprolide acetate (an LH-RH agonist), nafarelin and ganirelix. Also useful is a substance or metabolic precursor thereof, which is capable of promoting growth and survival of cells and tissues or augmenting the functioning of cells, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenetic protein, platelet-derived growth factor (PDGF), insulin-derived growth factor (IGF-I, IGF-II), transforming growth factor-alpha, transforming growth factor-beta, epidermal growth factor (EGF), fibroblast growth factor (FGF) and interleukin-1 (IL-1); an osteoinductive agent or bone growth promoting substance such as bone chips and demineralized freeze-dried bone material; and antineoplastic agents such as methotrexate, 5-fluoroacil, adriamycin, vinblastine, cisplatin, tumor-specific antibodies conjugated to toxins and tumor necrosis factor. Other useful substances include hormones such as progesterone, testosterone, and follicle stimulating hormone (FSH) (birth control, fertility-enhancement), insulin metal complexes and somatotropins; antihistamines such as diphenhydramine and chlorpheneramine; cardiovascular agents such as digitalis glycosides, papaverine and streptokinase; antiulcer agents such as cimetidine, famotidine and isopropamide iodide; vasodilators such as theophylline, B-adrenergic blocking agents and minoxidil; central nervous system agents such as dopamine; antipsychotic agents such as risperidone, olanzapine; narcotic antagonists such as naltrexone, maloxone and buprenorphine. Other examples of therapeutic and diagnostic agents are water insoluble anticancer drugs such as carmustine (BCNU), antiviral drugs such as azidothymidine (AZT) and other nucleosides, HIV Protease inhibitors such as saquinavir and retinovir immune-modulating agents such as cyclosporine, natural and synthetic hormones and hormone regulators such as contraceptives. Other therapeutic agents are steroidal and nonsteroidal anti-inflammatory agents such as hydrocortisone, prednisolone, ketoprofen, celecoxib and ibuprofen, centrally acting medicines such as antiseptics, antidepressants and sedatives and cardiovascular drugs such as anti-hypertensives and blood lipid lowering agents.

In another embodiment of the invention, the surfaces of the nanoparticles are modified, such as, for example by attaching a ligand to which a targeting agent is attached. Such ligands, and their attachment via standard conjugation chemistry, are known in the art [6]. For example, ligands, such as typical functional groups such as amino groups, carboxyl groups and sulfhydryl groups, are used. The targeting agent is an agent which is specific for an intended target. Such targeting agents include, for example, leutinizing hormone releasing hormone, growth hormone release hormone, epithelial growth factor, folic acid, antibodies specific for tumor markers, tumor specific drugs, and other targeting agents.

In another embodiment of the invention, additional paramagnetic MR contrast enhancing agents such as Gd-DTPA commonly used for H-1 MR imaging, can be incorporated into the nanoparticles to increase signal-to-noise-characteristics of the nanoparticles. Examples of such agents are included in U.S. Pat. No. 6,869,591, which is incorporated herein by reference.

Another aspect of the invention relates to the manufacture of the nanoparticles of the present invention. In this embodiment, the method includes providing a first liquid component of an emulsion system, providing a second liquid component of an emulsion system, providing a precursor, where the precursor is an alkoxy silane precursor which includes ¹⁹F, mixing the first liquid component, the second liquid component and the precursor, applying mechanical force to produce an emulsion which includes a dispersed phase and a continuous phase and separating the dispersed phase from the continuous phase to produce hybrid inorganic nanoparticles.

In one embodiment, the first liquid component is a surfactant. In one embodiment, the second liquid component is an acid.

Typical compounds which are used as the precursor in the method of the invention include all ¹⁹F alkoxy silane precursors. In one embodiment the precursor is 3,3,3-trifluoropropyl-trimethoxysilane (TFPTMS).

Typical surfactants include, for example, reaction products of natural or hydrogenated vegetable oils, and ethylene glycol; i.e., polyoxyethylene glycolated natural or hydrogenated vegetable oils, polyoxyethylene glycolated natural or hydrogenated castor oils, Cremophor RH-40, Cremophor RH60, Cremophor EL, Nikkol HCO-40, Nikkol HCo-60; Polyoxyethylene sorbitan fatty acid esters, e.g., mono- and tri-lauryl, palmityl, stearyl and oleyl esters; e.g. products of the trade name “Tween,” which includes polyoxyethylene sorbitan monolaurate (Tween), polyoxyethylene sorbitan mono-palmitate (Tween 40), polyoxyethylene sorbitan mono-oleate (Tween 80); Polyoxyethylene fatty acid esters, for example, polyoxyethylene stearic acid esters of the type known and commercially available under the trade name Myrj as well as polyoxyethylene fatty acid esters known and commercially available under the trade name Cetiol HE; Polyoxyethylene-polyoxypropylene co-polymers: e.g. of the type known and commercially available under the trade names Pluronic and Emkalyx; Polyoxyethylene-polyoxypropylene block co-polymers, of the type known and commercially available under the trade name Poloxamer; Dioctylsuccinate, dioctylsodiumsulfosuccinate, di-[2-ethylhexyl]-succinate, sodium lauryl sulfate; and Phospholipids, such as lecithins, for example, soybean lecithin; non-ionic polyoxyethylene fatty acid derivatives, such as polyoxyethylene sorbitan fatty acid esters (spans) such as sorbitan sesquiolate.

The mechanical force applied to the mixture includes any mechanical force known in the art to produce an emulsion, such as stirring. Separation of the dispersed phase and continuous phase is achieved by methods known to those skilled in the art, such as centrifugation. General methods for producing an emulsion system are described in [4], [6], and [12].

Optionally, the applying mechanical force step may be performed a number of times, for example, the method may include mixing the first liquid component with the precursor, followed by applying mechanical force, followed by adding the second liquid component, followed by, optionally, applying a second mechanical force step.

Mechanical force is applied for a period of from about 30 minutes up to about 15 hours, and all ranges in between, for example, from about 1 hour to about 6 hours, from about 2 hours to about 12 hours, from about 5 hours to about 15 hours. The mixing and applying mechanical force steps take place at about room temperature. The separation step takes place at about 2° to about 6° C.

Nanoparticles produced by the above method include an inner core and a surface and the ¹⁹F nuclei will be in the inner core of the nanoparticles.

In another embodiment of the invention, a second compound is added to the mixture. The addition of this compound results in additional amounts of ¹⁹F nuclei included in the nanoparticles of the invention. The additional amounts of ¹⁹F are provided by providing a second component, such as a perfluorocarbon, to incorporate additional amounts of ¹⁹F nuclei into the nanoparticles. In one embodiment a perfluorocarbon, such as zinc 1,2,3,4,8,8,9,10,11,15,16,17,18,22,23,24,25-hexadecafluoro-29H, 31H-phthalocyanine (ZnFP) is used.

In another embodiment of the invention, the ¹⁹F nuclei will be found either at the surface of the nanoparticles or at both the surface and in the inner core of the nanoparticles. For example, by preparing the nanoparticles by a reverse micellar method (using an organic solvent (like hexane, toluene etc.) as a bulk medium), the ¹⁹F nuclei will be on the outer surface of the nanoparticles.

The method of the present invention results in the production of nanoparticles having a size distribution of from about 20 to about 200 nanometers in diameter.

Another aspect of the invention relates to a method of imaging using the nanoparticles of the present invention. In the method, the nanoparticles of the present invention are administered to a subject and the subject is imaged. Using the nanoparticles of the present invention, an image, such as an MR image, having sufficient specificity and sensitivity is obtained.

Another aspect of the invention relates to a method of acquiring a spectroscopic acquisition of a subject. The method includes administering the nanoparticles of the present invention to the subject and obtaining a spectroscopic acquisition of the subject.

Another aspect of the invention relates to using the nanoparticles of the invention in other biomedical applications, such as a coating for medical devices, such as implantable medical devices such as, for example, stents, breast implants (to determine leakage or integrity of the implant), cardiac pacemakers, catheters or other implantable medical devices. Implantable medical devices refers to medical devices which are inserted into a subject.

### EXAMPLES

Magnetic resonance (MR) imaging is a noninvasive technique that has been applied to the detection, characterization and subsequent assessment of tumors and other soft tissue lesions following therapy. As it is commonly used, MR imaging utilizes the principles of nuclear magnetic resonance to obtain and decipher spectral patterns of ¹H (proton) magnetic resonance signals of body fluids and/or tissues. Typical ¹H images depict the distribution of water versus fat in a patient or sample. While ¹H MR imaging is arguably the best clinical diagnostic imaging modality available for non-invasive detection and characterization of in vivo tumors, several major drawbacks exist resulting in data yielding high resolution anatomic (structural) images of soft tissue but little physiologic (functional) information. In a similar fashion, other standard clinical diagnostic modalities suffer from the same drawback including computed tomography (CT), positron emission tomography (PET), X-ray, single photon emission computed tomography (SPECT) and ultrasound (US). Each modality can yield a plethora of either structural or functional information (albeit each with distinct advantages/disadvantages), but not a high degree of both during a single examination. The ability to readily provide researchers/clinicians with both structural and functional information during a single examination would significantly advance the field.

An alternative method of in vivo MR imaging is based on analysis of the spectral patterns of fluorine (¹⁹F) magnetic resonance signals, a non-radioactive species that is >99% naturally abundant and 83% as sensitive as ¹H. ¹⁹F MR imaging differs from ¹H MR imaging in that ¹⁹F nuclei are not naturally found in solution in living mammalian systems. Clinical applications of ¹⁹F MR imaging therefore will require specialized agents specifically designed for this purpose. However, in most other aspects, ¹⁹F MR is similar to standard ¹H techniques in terms of the imaging physics involved. Moreover, in vivo ¹⁹F MR imaging offers several advantages compared to ¹H based MR imaging methods. First, ¹⁹F containing compounds can be directly imaged by MR without background contamination from other molecules or anatomical structures. Secondly, ¹⁹F MR acquisitions yield images of the three-dimensional distribution of ¹⁹F containing molecules and therefore enable direct quantitative measurements of the biodistribution, pharmacokinetics and pharmacodynamics of administrated agents. Thirdly, for high resolution localization of ¹⁹F signals, images can subsequently be registered with high resolution ¹H MR images and/or acquired directly with arbitrarily high spatial resolution dependent only upon signal-to-noise (S/N) per unit time considerations (approx. 17% lower ¹⁹F S/N compared to ¹H S/N per molar concentration). Lastly, ¹⁹F MR T1 relaxation rates of many perfluorocarbon emulsions have been shown to correlate to pO2 concentrations in solution and in preliminary in vivo studies [1, 2]. This ability might allow for non-invasive measurement of tissue oxygenation before, during and after therapeutic intervention for assessing delivery of radiation, chemotherapy and/or photodynamic therapy (PDT) resulting in improved patient outcome.

Currently, the main limitation of ¹⁹F MR imaging is the paucity of available fluorine-containing compounds which can be administered in sufficient quantities for in vivo imaging while remaining non-toxic. To fill this void, silica nanoparticles containing an abundance of ¹⁹F molecules were specifically designed and synthesized as a platform for developing/optimizing ¹⁹F MR image acquisitions and for agent assessments to be used in a variety of biomedical applications including diagnostic applications, delivery of targeted therapies, as biomarkers or probes of tissue pO2 concentration, fiduciary markers for 3D registration, localization and visualization, molecular imaging of specific metabolic pathways, etc. Preliminary experiments have demonstrated the validity of this approach. Additionally, nanoparticles can encapsulate photosensitizing agents such as those typically used in photodynamic therapy (PDT) (e.g., 2-devinyl-2-(1-hexyloxyehtyl)pyropheophorbide commonly known as HPPH). Thus, the nanoparticle approach also represents a platform for the development of a new class of bifunctional agents that can be used for both therapy (e.g., PDT) and diagnostic assessment (e.g., ¹⁹F MR imaging) or as multimodality imaging probes to be used in fluorescence/bioluminescence and MR imaging exams. In vitro fluorescence imaging by confocal microscopy of HPPH doped silica nanoparticles has demonstrated that our nanoparticles are taken up by cancer cells in sufficient quantities so as to be imaged. Moreover, ¹⁹F nanoparticles can be concentrated and made to aggregate so as to yield a semi-solid crystalline or “slurry” containing little free water. In preliminary studies, strong ¹⁹F MR signal intensities were observed from these slurries that could be applied as biomedical “coatings” for assessing stent placement or as implantable “beads” for use in ¹⁹F-¹H MR image registration and/or as fiduciary markers for localization in 3D space and/or time. ¹⁹F MR imaging of “solid state” ¹⁹F containing materials has not been reported due to the generally short T2 relaxation times known for other ¹⁹F containing solids [3] (e.g., Teflon®). For example, if T2 relaxation times occur in timeframes shorter than what can be observed using MR pulse acquisition sequences commonly employed for imaging, then no MR image can be constructed from the raw data. In summary, the ¹⁹F nanoparticles of the present invention could have an impact on medical imaging and facilitate the development of new multimodality based imaging methods. In a manner analogous to the introduction of iodinated contrast media originally developed over 100 yrs. ago and still in use today to enhance X-ray image contrast in clinical practice, silica based ¹⁹F nanoparticles could significantly impact medical imaging and change the manner in which clinical medicine is currently practiced.

### Example 1

### Synthesis and Characterization of Dye Loaded Silica Based TFPTMS Nanoparticles

Silica based nanoparticles containing ¹⁹F nuclei using a precursor 3,3,3-trifluoropropyl-trimethoxysilane (TFPTMS) were synthesized. Silica based ¹⁹F nanoparticles loaded, with a porphyrin based zinc compound (zinc 1,2,3,4,8,8,9,10,11,15,16,17,18,22,23,24,25-hexadecafluoro-29H, 31H-phthalocyanine) containing 60 ¹⁹F nuclei, were synthesized either in-polar core of Aerosol-OT/DMSO/water microemulsions or Tween-80/DMSO/water microemulsion. The loaded and unloaded nanoparticles were prepared by using the following methods:

A) Preparation of Void TFPTMS Nanoparticles

In a typical experiment, the micelles were prepared by mixing 3.0 ml of butanol-1 and 500 μl DMSO to 100 ml of 2% Tween-80 solution in double distilled water with the help of a magnetic stirrer. After half an hour stirring, 1 ml of the neat TFPTMS was added and stirred vigorously for 3-5 hrs. Finally, 2 mL hydrochloric acid (˜6.0 N) solution was added and stirred overnight. At the end of the process, a white translucency indicating the formation of nanoparticles was observed. The next day the nanoparticles were separated out by centrifugation at 11000 rpm (at 4° C.) for one hour. Further, the centrifuged nanoparticles were washed at least three times with double distilled water to remove the unreacted materials.

B) Preparation of Loaded TFPTMS Nanoparticles

In case of Zinc 1,2,3,4,8,8,9,10,11,15,16,17,18,22,23,24,25-hexadecafluoro-29H,31H-phthalocyanine (ZnFP) loaded nanoparticles, the micelles were prepared by dissolving a 2.2 g of AOT (sodium bis-2-ethylhexyl-sulfosuccinate) and 4.0 ml 1-butanol in 100 ml of double distilled water by vigorous magnetic stirring. A 500 μl sample of zinc 1,2,3,4,8,8,9,10,11,15,16,17,18,22,23,24,25-hexadecafluoro-29H,31H-phthalocyanine in dimethyl sulfoxide (DMSO) (10 mM) was dissolved in the above solution by magnetic stirring. After that, 1.0 ml of neat 3,3,3-trifluoropropyltrimethoxysilane (TFPTMS) was added to the micellar system, and the resulting solution was stirred for about 3-5 hours. Next, nanoparticles were precipitated by adding 1.5 ml of hydrochloric acid (˜6N) solution stirring for about 72 hours. The entire reaction was carried out at room temperature. The nanoparticles were separated out by centrifuging at 11,000 rpm (4° C.) for at least one hour. The main object of doping the zinc 1,2,3,4,8,8,9,10,11,15,16,17,18,22,23,24,25-hexadecafluoro-29H,31H-phthalo-cyanine is to increase the concentration and subsequent ¹⁹F signal-to-noise in MR imaging experiments.

### Example 2

### Determination of the Size and Morphology of the Nanoparticles

Size and the morphology of TFPTMS nanoparticles as produced in Example 1 were examined by using Transmission Electron Microscope (TEM). After completion of the synthesis process, one drop of this TFPTMS (at least 5 times dilutes) was mounted on a thin film of pure carbon deposited on a copper grid. The grid was then examined under an electron microscope (model JEOL 2010 microscope). Nanoparticles size distribution was found to be approx. 10-20 nm in diameter and generally spherical in shape (not shown).

### Example 3

### ¹⁹F NMR Spectra

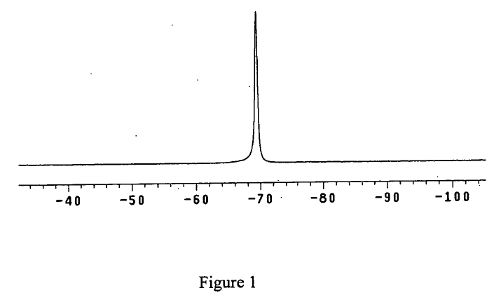

Silica based TFPTMS nanoparticles as produced in Example 1 were characterized by ¹⁹F-NMR spectroscopy by suspending a small quantity in 90% D₂O and acquiring ¹⁹F-NMR spectra using a Varian Inova-400 NMR Spectrometer (Varian, Palo Alto, Calif.) operating at 376.3 MHz for ¹⁹F nucleus. The data were fourier transformed (FT) with an exponential function and expressed to ¹H at 0.0 ppm relative to tetramethoxy silane (TMS) at room temperature. The results are as shown in FIG. 1.

### Example 4

### In Vitro Fluorescence Imaging

For in vitro fluorescence imaging, the photosensitizer, (2-devinyl-2-(1-hexyloxyehtyl)pyropheophorbide, (HPPH), was used. Although any appropriate hydrophobic fluorescence dye could be incorporated in nanoparticles of the present invention, HPPH was chosen for demonstration purpose only. HPPH doped nanoparticles were prepared by the technique described above in Example 1 except here 50 μl of HPPH (8 mg/ml DMSO) was added and in a smaller scale. Thus, in a typical experiment, 0.22 g of AOT was dissolved by adding 10 ml of distilled water and 400 μl of butanol-1 by vigorous stirring. Fifty μl of HPPH (8 mg/ml DMSO) was added, followed by the addition of 100 μl of 3,3,3-trifluoropropyl-trimethoxysilane, and the whole mixture was stirred for at least two hours. Then, 150 μl of HCl (˜6N) was added for the hydrolysis of 3,3,3-trifluoropropyl-trimethoxysilane for at least 72 hours resulting in the formation of silica based TFPTMS ¹⁹F nanoparticles. Next, the surfactant and free dye were removed by dialysis against water for 50 hours. The dialyzed solution was filtered though 0.22 μm filters membrane for use in imaging experiments. It was also seen that by using Tween-80 as a surfactant instead of AOT hydrophilic dye, hydrophobic agents like HPPH, can be incorporated. For demonstrating imaged based nanoparticle uptake into cells, three different tumor cell lines were employed and studied using cell culture protocols. The cell lines used were UCI-107 (Uterine Carcinoma), MCF-7 (Human breast cancer) and HepG2 (human hepatocarcinoma). For in vitro fluorescence imaging, cells were first trypsinized and resuspended in suitable culture medium at a concentration of 7.5×10⁵ per ml. Approximately 0.10 ml of this cell suspension was combined with 5 ml of medium on 60 mm culture plates followed by overnight incubation at 37° C. with 5% CO₂ in an incubator (VWR Scientific model 2400, Bridgeport, N.J.). After overnight incubation, the cells were rinsed with Phopshate-Buffered Saline (PBS) and 5 ml of fresh medium was added to it. Subsequently, 100 μl of the dialysed HPPH doped silica based TFMPTS nanoparticles which were filtered through 0.22 μm syringe filter membrane were added to each plate and thoroughly mixed. Then, the HPPH doped silica based TFMPTS nanoparticles treated cells were again incubated in the same incubator (37° C. with 5% CO₂) for at least one hour. The incubated cells were again rinsed with PBS and 5 ml of fresh medium was added to prepare the cells ready for imaging. The cells were then directly imaged using a confocal laser scanning microscope (MRC-1024, Bio-Rad, Richmond, Calif.), which was attached to an upright (Nikon model Eclipse E800) camera. Further, localized spectroflurometry on the cells [4] ensured that the observed fluorescence was from HPPH doped silica based TFMPTS nanoparticles. Thus, from in vitro fluorescence results, it is clear that HPPH containing nanoparticles entered tumor cells in sufficient quantities so as to be imaged in all cases (HepG2, MCF-7 and UCI-107).

### Example 5

### In Vitro ¹⁹F MR Imaging and Spectroscopy

High resolution in vitro ¹⁹F MR spectra of the silica based TFPTMS nanoparticles were acquired using a General Electric (GE) CSI 4.7T/33 cm horizontal bore magnet (GE NMR Instruments, Fremont, Calif.) operating at 188.342705 MHz for ¹⁹F using radio-frequency (RF) and computer systems incorporating AVANCE digital electronics (Bruker BioSpec platform with Paravision® Version 3.01 Operating System; Bruker BioSpin MRI, Billerica, Mass.). MR data (spectra and images) were acquired using a G060 removable gradient coil insert generating a maximum field strength of 950 mT/m and a custom-designed 35 mm RF transceiver coil serially tuned to ¹H or ¹⁹F resonances (Bruker Biospin, Billerica, Mass.).

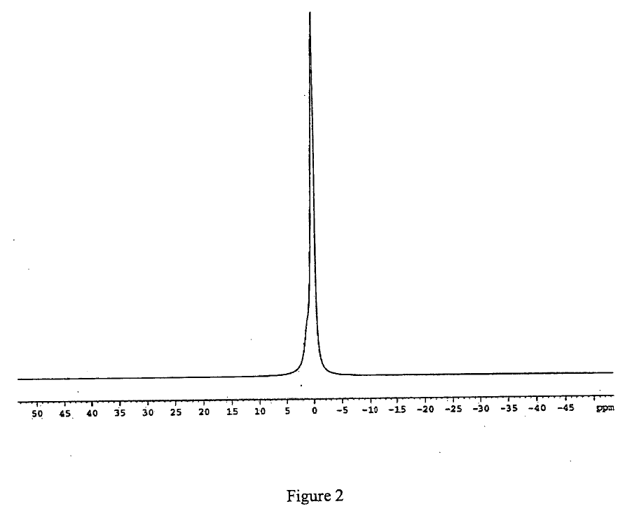

¹⁹F MR spectra were acquired from neat nanoparticle preparations immediately before imaging by first frequency tuning and impedance matching our RF transceiver coil to the resonance frequency of ¹⁹F nuclei. A RF, non-slice selective 90° block pulse was applied and magnetic field shimming performed to optimize magnetic field homogeneity over the entire sample. Transmit and receiver gains were then determined for slice selective 90° to 180° and results used to optimize S/N relationships in resultant data sets. ¹⁹F MR spectra were obtained using a RF non-slice selective 90° block pulse or a slice selective 90° sinc3 RF pulse. Typical acquisition parameters consisted of 1-16 NEX (number of excitations) and were acquired in 1-2 min. A typical MR spectra is shown in FIG. 2.

¹⁹F MR images were acquired using standard 2D or 3D spin echo (SE), rapid acquisition with refocused echoes (RARE) SE or gradient recalled echo (GRE) MR imaging pulse sequences. A typical MR image acquisition consisted of a series of scans in the axial, sagittal and/or coronal plane including a localizer, T1-weighted SE (or proton-density-weighted) and T1-weighted RARE SE MR images. Typical acquisition parameters consisted of 6-30 mm thick slices with a 3.2×4.8 cm field of view (FOV), 64×64 matrix, 1-16 NEX, 1-16 slices using TRITE (time for repetition/time for echo)=1200/14 ms for T1-weighted SE acquisitions, TR/TE=2000/20-41 ms with an echo train=4 or 8 for more proton-density-weighted RARE acquisitions. A representative ¹⁹F MR image of silica based TFPTMS nanoparticles was obtained (not shown). The composite ¹⁹F MR image of two separate MR acquisitions clearly demonstrated a direct relationship between ¹⁹F MR signal intensity and ¹⁹F concentration. A sagittal acquisition-depicted seven 200 μl wells containing increasing amounts of neat silica based TFPTMS nanoparticles. A coronal acquisition fully encompassing the 200 μl wells in the sagittal image were acquired using identical MR parameters. Results from a line profile through coronal image demonstrated that a linear increase in signal intensity as concentration of neat silica based TFPTMS nanoparticles is linearly increased. Unlike ¹H MR images, this demonstrates that ¹⁹F contrast agents offer an easily quantifiable metric of ¹⁹F concentration of labeled agents. ¹H MR acquisitions obtained using FDA approved MR contrast enhancing agents employ paramagnetic metal ions to induce non-linearly increased ¹H S/N per unit time in regions containing the ions on T1-weighted MR acquisitions [5]. Because the paramagnetic metal ion's effect on proton relaxation is measured indirectly (i.e., proton relaxation, not Gd concentration, is measured), absolute measurement of Gd-labeled contrast enhancing agent concentration is complex, often ambiguous and confounded by physiologic processes. ¹⁹F MR images employing ¹⁹F labeled agents do not suffer from these disadvantages.

### Example 6

### ¹⁹F Spectra Obtained from Neat Silica Based TFPTMS ¹⁹F Containing Nanoparticles and NaF in Aqueous Solution

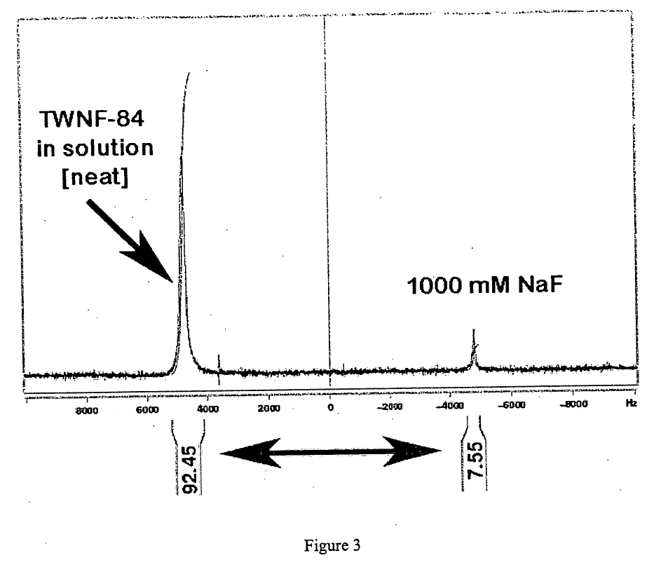

¹⁹F spectra obtained from two vials (placed symmetrically around magnetic field isocenter) containing equal volumes of either neat silica based TFPTMS ¹⁹F nanoparticles or 1000 mM NaF in aqueous solution is shown in FIG. 3. Clearly shown is the dramatically increased S/N per unit volume per unit time from the ¹⁹F labeled nanoparticles compared to NaF acquired using a 90° block pulse with a center frequency approx. midway between their resonant frequencies. Integrated peak intensities as shown were 92.45 versus 7.55 relative units. Similarly, when subsequent spectral acquisitions were obtained by shifting the center frequency of the 90° block pulse to each of the resonance peaks in separate data acquisitions maintaining all other MR parameters identical, results for signal to noise measurements were as follows: S/N=783 for silica based TFPTMS ¹⁹F nanoparticle versus S/N=27.3 for 1000 mM NaF in aqueous solution. This represents a 28.8 fold relative increase in MR sensitivity for the silica based nanoparticles as compared to 1000 mM NaF compared on an equal volume basis. Moreover, this figure demonstrates the significant increase in dynamic range in ¹⁹F chemical shift for ¹⁹F labeled agents (6,000-12,000 Hz at 4.7 T) that can be used as a sensitive probe to study specific ¹⁹F species (metabolic, catabolic processes) as compared to ¹H chemical shifts (typically 200-800 Hz at 4.7 T).

### Example 7

### T1, T2 MR Relaxation Time Experiments

T1 and T2 relaxation times are phenomenologically defined time constants commonly used in MR to describe the regrowth of longitudinal magnetization (T1) along the z axis or the decay of magnetization of the transverse components (T2) along the x-y plane after application of a RF pulse. Knowledge of T1 and T2 relaxation times can be used to determine and optimize signal-to-noise characteristics and image contrast in MR data acquisitions. T1 relaxation rates (1/T1 relaxation time=R1 relaxivity) were acquired for a range of contrast agent concentrations using a saturation recovery SE sequence with a fixed TE=10 ms and TR times ranging from 52 to 6000 ms (FOV=32×32 mm, slice thickness=8 mm, slices=1, matrix=64×64, NEX=2. Signal intensities at each repetition time were obtained by taking the mean intensity within a region of interest (ROI) and R1 and SDs determined by nonlinear fitting of the equation: Y=A(1−exp(−TR/T1)) using software provided by the manufacturer. Similarly, T2 relaxation rates (R2) were acquired using a multi-echo, CPMG SE sequence with a fixed TR of 2760 ms and TE times ranging from 8.21 to 164.2 ms. R2 and SDs were determined as described above using the equation: Y=A+C*exp(−TE/T2). T1 relaxation time for void nanoparticles preparation at 188.342705 MHz for ¹⁹F was determined to be approx. 482.9 ms while T2 relaxation time was determined to be approx. 14.7 ms. In general, short T1 relaxation times with moderately short T2 relaxation times similar to those obtained herein yield high MR signal intensities per unit time on T1-weighted MR acquisitions (i.e., short TE, short to moderate TR MR acquisition times).

### Example 8

### In Vivo ¹⁹F MR Imaging

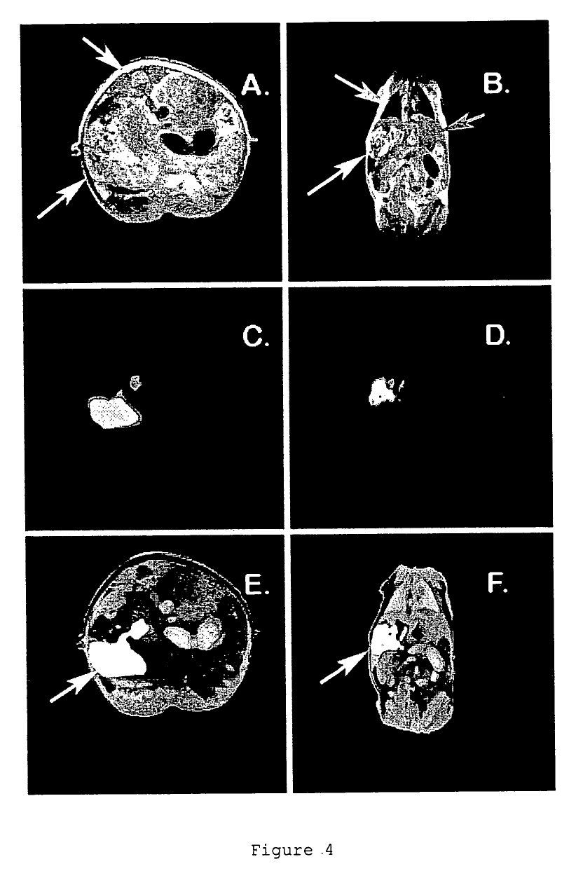

High resolution in vivo ¹⁹F MR images of the silica based TFPTMS nanoparticles were acquired using a General Electric (GE) CSI 4.7T/33 cm horizontal bore magnet (GE NMR Instruments, Fremont, Calif.) operating at 188.342705 MHz for ¹⁹F using radio-frequency (RF) and computer systems incorporating AVANCE digital electronics (Bruker BioSpec platform with Paravision® Version 3.01 Operating System; Bruker BioSpin, Billerica, Mass.). MR data (spectra and images) were acquired using a G060 removable gradient coil insert generating a maximum field strength of 950 mT/m, a custom-designed 35 mm RF transceiver coil serially tuned to ¹H or ¹⁹F resonances (Bruker BioSpin, Billerica, Mass.), for standard spin echo (SE), and rapid acquisition with relaxation enhancement (RARE) SE MR imaging pulse sequences. A typical acquisition consisted of a series of scans including ¹H and ¹⁹F localizer images, T1-weighted SE and/or RARE SE MR images spanning the entire liver, upper and lower abdomen. Coronal and axial ¹H and ¹⁹F images were routinely acquired for murine imaging. Briefly, mice were administered the nanoparticle preparation orally (po) by gavage and anesthetized for imaging by injection of 100 mg/kg ketamine HCl+10 mg/kg xylazine via intraperitoneal (ip) injection. Typical MR acquisition parameters consisted of 3 mm thick slice(s) for ¹H or 15-30 mm thick slice(s) for ¹⁹F acquisitions with a 32 mm×32 mm field of view (FOV) for axial acquisitions or 64 mm×32 mm FOV for coronal acquisitions, 128×128 matrix for ¹H or 64×64 matrix for ¹⁹F acquisitions, 1-4 NEX, 1-12 slices using TR/TE=424/10 ms for T1-weighted ¹H SE acquisitions or TR/TE=1400/8.5 ms for T1-weighted ¹⁹F SE acquisitions. A series of ¹H and ¹⁹F MR murine images (FIG. 4) were obtained immediately after oral administration of silica based TFPTMS nanoparticles. Note: ¹⁹F MR signal intensities in images C and D were obtained only from regions containing nanoparticles (stomach). ¹H images (A and B) were 1 mm thick slices acquired approximately midline through mouse in either the axial or coronal plane while ¹⁹F MR images (C and D) were approximately 30 mm thick (analogous to an X-ray image or projection through the mouse) acquired with identical spatial registration parameters, but with a 64×64 matrix (¹⁹F) versus 256×256 matrix (1H). Panels E and F depict a summary of ¹H and ¹⁹F data demonstrating the spatial localization of the ¹⁹F MR signal obtained from the nanoparticles. Briefly, the look-up-table (LUT) for the grey scale images (as shown in A and B) were inverted and fused with ¹⁹F acquired data (as shown in panels C and D). ¹⁹F signal intensity values were then modified to a grey-scale value of 255 for increased conspicuity (0-255 level 8 bit image).

### Example 9

### “Solid State” ¹⁹F MR Imaging of Semi-Solid Crystalline Aggregates

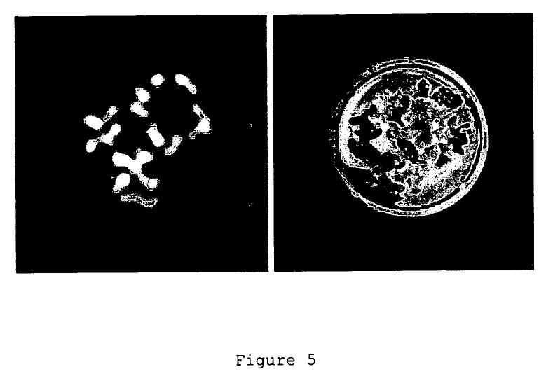

High resolution in vivo ¹⁹F MR images of the silica based TFPTMS nanoparticles doped with ZnPF were acquired as previously described for in vitro and in vivo MR acquisitions using standard SE and RARE SE MR imaging pulse sequences. A typical acquisition consisted of a series of scans including ¹H and ¹⁹F localizer images, T1-weighted SE and/or RARE SE MR images in the coronal and axial ¹H and ¹⁹F images. Typical MR acquisition parameters consisted of 3 mm thick slice(s) for ¹H or 15-30 mm thick slice(s) for ¹⁹F acquisitions with a 32 mm×32 mm field of view (FOV) for axial acquisitions or 64 mm×32 mm FOV for coronal acquisitions, 128×128 matrix for ¹H or 32×32 matrix for ¹⁹F acquisitions, 32 NEX, 1-12 slices using TR/TE=424/10 ms for T1-weighted ¹H SE acquisitions or TR/TE_{eff}=2045/22.5 ms for moderately T1-weighted ¹⁹F SE acquisitions. ¹⁹F MR images (FIG. 5) (a) of semi-solid crystalline aggregates of silica based TFPTMS ¹⁹F containing nanoparticles doped with ZnFP obtained from the same sample photographed in (b) and shown in the same general orientation. The nanoparticles in the bottom of the glass tube were photographed using a surgical microscope with attached Nikon 1.2 Mb digital camera (Nikon CoolPix 950 camera, Nikon USA).

### Example 10

### Toxicity

In preliminary studies, no significant acute toxicity due to the silica based TFPTMS ¹⁹F containing nanoparticles was observed when administered to a small animal model of disease.

Discussion

A number of researchers and manufacturers have been trying to develop image based agents to improve the sensitivity and specificity of MR and other imaging modalities such as CT, PET, SPECT, US while maintaining high spatial and temporal resolution as well as structural, functional relationships [7, 8, 9]. To date, this has not been feasible, demonstrated or proposed. The ultimate goal is to obtain the specificity and sensitivity already demonstrated from optical based methods including bioluminescence, fluorescence and near infrared (near IR) imaging typically used in cell culture studies employing a gamut of available probes such as green fluorescent protein (GFP), red fluorescent protein (RFP) and other fluorophores [10]. However, the major inherent limitation of optical based methods at the present time appears to be inherent light scattering artifacts which severely limit the depth of penetration of the excitation and/or transmission of light in biological systems [11]. Due to the inherent physics of the problem, overcoming these limitations may not be possible.

In theory, ¹⁹F MR imaging techniques coupled to current ¹H MR methods can overcome these barriers and could significantly impact current practices. The major drawback currently facing the commercialization and clinical application of ¹⁹F MR techniques concerns the lack of a suitable ¹⁹F containing probe that can be administered in sufficient quantities without subsequent toxicity. In this regard, the synthesis, application and further development of silica based TFPTMS ¹⁹F containing nanoparticles and other similarly labeled nanoparticles as a platform for delivering ¹⁹F nuclei in sufficient quantities represents a significant advance that could facilitate additional novel applications and discoveries. Additional increases in S/N are possible and expected in the near future using improved MR hardware and software instrumentation as well as modification and optimization of our nanoparticles.

Presently, non-invasive image based methods to accurately assess pO2 values in tissue do not exist. While some recent developments appear promising (e.g., near IR tomographic imaging of fluorescent probes designed for this purpose), a clear void in this area currently exists. The ability to non-invasively assess pO2 in tumors and other tissues in near real-time would permit near real-time optimization of radiation, chemo and/or photodynamic therapy dose delivery leading to improved prognostic indicators of treatment. Silica based TFPTMS ¹⁹F containing nanoparticles as a semi-solid crystalline aggregate can be readily imaged and used as a “surface coating” or embedded within other materials for 2D, 3D spatial localization of medical devices or as a fudiciary marker for image registration or potentially as a calibration standard for quality assurance testing. Currently no solid state calibration standard exists for MR and only “relative” changes in MR signal intensity at specific magnetic field strengths and pulse sequences are used. This limitation represents another major disadvantage of current MR instrumentation, i.e., it is difficult or impossible to compare absolute MR signal intensities acquired on one MR system to those obtained on a different MR system or the same system at a different points in time.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### REFERENCES

1. Krafft, M P. Fluorocarbons and fluorinated amphiphiles in drug delivery and biomedical research. Advanced Drug Delivery Reviews. Vol. 47. 209-228, 2001.
2. McIntyre, D J O, McCoy, C L, and Griffiths, J R. Tumour oxygenation measurements by 19F magnetic resonance imaging of perfluorocarbons.
3. Randall, E W. ¹H and ¹⁹F magnetic resonance imaging of solid paramagnetic compounds using large magnetic field gradients and Hahn echoes. Solid State Nucl Magn Reson. 1997 May; 8(3):173-8.
4. Roy I, Ohulchansky T Y, Pudavar H E, Bergey, J E, Oseroff A R, Morgan J, Dougherty T J, Prasad, P N. Ceramic based nanoparticles entrapping water insoluble photosensitizing anticancer drugs: A novel drug carrier system for photodynamic therapy. J. Am. Chem. Soc. 125, 7860-7865, 2003.
5. Weinmann H J, Brasch R C, Press W R, Wesbey G E. Characteristics of gadolinium-DTPA complex: a potential NMR contrast agent. AJR Am J Roentgenol. 1984 March; 142(3):619-24.
6. Roy I, Ohulchanskyy T Y, Bharali D J, Pudavar H E, Mistretta R A, Kaur N, Prasad P N. Optical tracking of organically modified silica nanoparticles as DNA carriers: a nonviral, nanomedicine approach for gene delivery. Proc Natl Acad Sci SA 102 (2):279-284 2005.
7. Blasberg, R G. Molecular imaging and cancer. Molecular Cancer Therapeutics. Vol. 2, 335-343. 2003.
8. Neeman M, and Dafni, H. Structural, functional and molecular MR imaging of the microvasculature. Annu. Rev. Biomed. Eng. Vol. 5, 29-56, 2003.
9. Weissleder R, and Mahmood, U. Molecular imaging/Radiology. Vol. 219: 316-333, 2001.
10. Carrington, C. Optical imaging shed light on cancer's signature. Diagnostic Imaging. June 2004.
11. Choy G, O'Connor S, Diehn F E, Costouros N, Alexander H R, Choyke P, Libutti S K. Comparison of noninvasive fluorescent and bioluminescent small animal optical imaging. Biotechniques. 2003 November; 35(5):1022-6, 1028-30.
12. Bharali D J, Klejbor I, Stachowiak E K, Dutta P, Roy I, Kaur N, Bergey E J, Prasad P N, Stachowiak M K. Organically modified silica nanoparticles: a nonviral vector for in vivo gene delivery and expression in the brain. Proc Natl Acad Sci USA 2005 Aug. 9: 102(32): 279-84.

What is claimed is:

1. A hybrid inorganic nanoparticle which includes from about 2000 to about 600,000 ¹⁹F nuclei.

2. A hybrid inorganic nanoparticle according to claim 1 wherein the nanoparticle comprises from about 10,000 to about 600,000 ¹⁹F nuclei.

3. A hybrid inorganic nanoparticle according to claim 1 wherein the nanoparticle comprises from about 100,000 to about 600,000 ¹⁹F nuclei.

4. A hybrid inorganic nanoparticle according to claim 1 wherein the nanoparticle comprises from about 300,000 to about 600,000 ¹⁹F nuclei.

5. A hybrid inorganic nanoparticle according to claim 1 wherein the nanoparticle is a silica based hybrid inorganic nanoparticle.

6. A hybrid inorganic nanoparticle according to claim 1 further comprising a florescent dye, a bioluminescent marker, a near infrared marker, a therapeutic agent, a diagnostic agent, a targeting agent or a paramagnetic contrast enhancing agent.

7. A hybrid inorganic nanoparticle according to claim 1 wherein the nanoparticle is from about 20 to about 200 nm in diameter.

8. A hybrid inorganic nanoparticle according to claim 1 wherein the nanoparticle is from about 50 to about 200 nm in diameter.

9. A method of making hybrid inorganic nanoparticles, the method comprising:
providing a first liquid component of an emulsion system;
providing a second liquid component of an emulsion system;
providing a precursor, wherein the precursor is an alkoxy silane precursor which includes ¹⁹F;
mixing the first liquid component, the second liquid component and the precursor;
applying mechanical force to produce an emulsion which includes a dispersed phase and a continuous phase; and
separating the dispersed phase from the continuous phase to produce the hybrid inorganic nanoparticles, wherein the nanoparticles are from about 20 nm to about 200 nm in diameter and comprise ¹⁹F nuclei.

10. A method according to claim 9, further comprising providing a perfluorocarbon and mixing the perfluorocarbon with the first liquid component, the second liquid component the precursor, or any combination thereof.

11. A method according to claim 9 wherein the precursor is 3,3,3-trifluoropropyl-trimethoxysilane.

12. A method according to claim 10 wherein the perfluorocarbon is zinc 1,2,3,4,8,8,9,10,11,15,16,17,18,22,23,24,25-hexadecafluoro-29H,31H-phthalocyanine and the precursor is 3,3,3-trifluoropropyl-trimethoxysilane.

13. A method according to claim 9 further comprising adding a florescent dye, bioluminescent marker, near infrared marker, diagnostic agent, or therapeutic agent to the mixture, whereby the nanoparticles comprise the florescent dye, bioluminescent marker, near infrared marker, diagnostic agent, therapeutic agent.

14. A method according to claim 9 wherein the nanoparticles comprise from about 10,000 to about 600,000 ¹⁹F nuclei per nanoparticle.

15. A method according to claim 9 wherein the nanoparticles comprise from about 100,000 to about 600,000 ¹⁹F nuclei per nanoparticle.

16. A method according to claim 15, wherein the nanoparticles are from about 40 nm to about 200 nm in diameter.

17. A method according to claim 9 further comprising modifying the surface of the nanoparticles to attach a targeting agent.

18. A method of imaging comprising:
administering a plurality of hybrid inorganic nanoparticles according to claim 1 to a subject and
imaging the subject.

19. A method of acquiring a spectroscopic acquisition of a subject comprising:
administering the nanoparticles of the present invention to the subject and
obtaining a spectroscopic acquisition of the subject.

20. An implantable medical device comprising a plurality of nanoparticles according to claim 1.
